(19) United States
(12) Patent Application Publication
(10) Pub. No.: US 2016/0235512 A1
(43) Pub. Date: Aug. 18, 2016

(54) ANIMAL INTRANASAL ADMINISTRATION DEVICE, SYSTEMS, AND ASSOCIATED METHODS

(71) Applicant: Bovicor Pharmatech Inc., Vancouver (CA)

(72) Inventors: Christopher C. Miller, North Vancouver (CA); Gilly Regev-Shoshani, Vancouver (CA); Alex Stenzler, Long Beach, CA (US); Steve Han, Huntington Beach, CA (US)

(73) Assignee: Bovicor Pharmatech Inc., Vancouver (CA)

(21) Appl. No.: 15/003,566

(22) Filed: Jan. 21, 2016

Related U.S. Application Data
(60) Provisional application No. 62/106,173, filed on Jan. 21, 2015.

(51) Int. Cl. A61D 7/04; A61K 33/00; A61K 9/00

(52) U.S. Cl. CPC A61D 7/04; A61K 9/0043; A61K 33/00

(57) ABSTRACT

An animal intranasal administration device, and associated systems and methods, are disclosed. The animal intranasal administration device can include a nasal passage nozzle for each nostril configured to receive fluid from a fluid source. The animal intranasal administration device can also include a biasing mechanism to bias the nozzles toward a septum such that the device is secured in place about the septum during administration of the fluid into nasal passages.

### PRIORITY DATA

This application claims the benefit of U.S. Provisional Patent Application Ser. No. 62/106,173, filed Jan. 21, 2015, which is incorporated herein by reference.

### BACKGROUND OF THE INVENTION

Nitric oxide gas has an antimicrobial effect and when safely administered can be used as a therapeutic treatment of microbial infection in a subject. While many systems have been described for the use of nitric oxide in clinical settings, these systems are designed for the delivery of nitric oxide gas to the subject in a way that requires the subject to remain stationary for an extended period of time. Unfortunately, many instances where treatment of nitric oxide would be particularly beneficial do not allow for the subject to be stationary or immobilized for the length of time needed to receive an effective dosage of nitric oxide gas.

For example, one such instance is in the cattle industry, where Bovine Respiratory Disease Complex (BRDc) continues to be the most common disease in feeder beef cattle in North America, effecting 20-40% of receiver calves annually. Production losses from BRDc include respiratory morbidity and mortality as well as increased treatment and processing cost. Its pathogenicity has been linked to a primary viral infection followed by a secondary bacterial infection.

While the incidence of BRDc has been shown to be reduced in animals treated with a suitable dosage of nitric oxide gas, effective commercialization of such therapy remains infeasible due to administration time constraints.

### SUMMARY OF THE INVENTION

Although nitric oxide gas can be used to treat animals with BRDc, such treatment typically requires 30 minutes of exposure to be effective, which is a very significant amount of time during commercial operations and difficult to implement. Accordingly, the present inventors have recognized a need for a device, system, and method to quickly and efficiently deliver an effective dose of a nitric oxide gas. In one embodiment, the dosage can be quickly administered, but provide the benefit and activity of a long acting and effective nitric oxide gas dose. By way of example, without limitation, a nitric oxide releasing solution (NORS) can be administered quickly and then release nitric oxide (NO) gas in-vivo for an extended or prolonged duration. In another example, an NO gas (gNO) can be delivered directly from the present devices as a gas per se and not as a NORS.

In one aspect, devices for administration of nitric oxide gas (including from NORS) is provided. In one embodiment, administration can occur intranasally. In some embodiments, the intranasal administration device can be customized for administration to a particular animal or subject. For example, an animal intranasal administration device can include a nasal passage nozzle for each nostril configured to receive fluid from a fluid source. The intranasal administration device can also include a biasing mechanism to bias the nozzles toward a septum such that the device is secured in place about the septum during administration of the fluid into nasal passages.

The present invention also provides an animal intranasal administration device that can include a support member having a first support member portion and a second support member portion. The animal intranasal administration device can also include a first nasal passage nozzle coupled to the first support member portion and a second nasal passage nozzle coupled to the second support member portion. The first support member portion and the second support member portion can be movable relative to one another to secure the first and second nasal passage nozzles at least partially within nostrils of an animal about a septum and such that fluid is directed into nasal passages of the animal. One benefit thusly provided is that the NO can be delivered by the device without the necessity of a user to hold the device in place during administration, and allows them to have their hands free to perform other tasks.

Additional invention embodiments encompass an animal intranasal administration system. In some aspects, such a system can include an animal intranasal administration device as recited herein, for example, that includes a support member having a first support member portion and a second support member portion. The animal intranasal administration device can also include a first nasal passage nozzle coupled to the first support member portion and a second nasal passage nozzle coupled to the second support member portion. The first support member portion and the second support member portion can be movable relative to one another to secure the first and second nasal passage nozzles at least partially within nostrils of an animal about a septum and such that fluid is directed into nasal passages of the animal. The system can also include a fluid source fluidly coupled to each of the first and second nasal passage nozzles.

Yet additional invention embodiments encompass methods of administering a fluid to an animal's nostril. The method can include providing an animal intranasal administration device and/or system as recited herein, for example that can include a support member having a first support member portion and a second support member portion. The animal intranasal administration device can also include a first nasal passage nozzle coupled to the first support member portion and a second nasal passage nozzle coupled to the second support member portion. The first support member portion and the second support member portion can be movable relative to one another to secure the first and second nasal passage nozzles at least partially within nostrils of an animal about a septum and such that fluid is directed into nasal passages of the animal. The method can also include engaging the device with the animal's nostril. Additionally, the method can include dispensing the fluid from the device and into the animal's nostrils.

There has thus been outlined, rather broadly, various features of the invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a schematic illustration of an animal intranasal administration system, in accordance with an example of the present disclosure.

FIG. 2A is a schematic illustration of an animal intranasal administration system, in accordance with another example of the present disclosure.

FIG. 2B is a schematic illustration of an animal intranasal administration system, in accordance with yet another example of the present disclosure.

FIG. 2C is a schematic illustration of an animal intranasal administration system, in accordance with still another example of the present disclosure.

FIG. 3A is a perspective view of an animal intranasal administration device, in accordance with an example of the present disclosure.

FIG. 3B is a bottom view of the animal intranasal administration device of FIG. 3A engaged with a septum of an animal.

FIG. 3C is a side view of the animal intranasal administration device of FIG. 3A engaged with a septum of an animal.

FIG. 4 is an isolated view of animal intranasal administration device spray heads, in accordance with an example of the present disclosure.

FIG. 5 is a perspective view of an animal intranasal administration device, in accordance with another example of the present disclosure.

FIGS. 6A-6C illustrate aspects of an animal intranasal administration system, in accordance with a further example of the present disclosure.

### DETAILED DESCRIPTION OF THE INVENTION

It is to be understood that the figures and descriptions of the present invention have been simplified to illustrate elements that are relevant for a clear understanding of the present invention, while eliminating, for the purpose of clarity, many other elements found in typical nitric oxide delivery formulations and delivery systems. Those of ordinary skill in the art may recognize that other elements and/or steps are desirable and/or required in implementing the present invention. However, because such elements and steps (e.g. analyzers with alarms to monitor methemoglobin and/or environmental safety) are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a,” “an,” and “the” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate. It is to be understood that in the present specification, the use of the term “about” in connection with a numerical value also affords support for the exact numerical value as though it had been recited without the term “about”.

In this specification, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

“NORS” as used herein may refer to a nitric oxide releasing solution or substance. In one aspect, NO released from NORS may be a gas.

As used herein, “gaseous nitric oxide,” or “gNO” refers to exogenous gastric oxide. gNO can be delivered to a veterinary subject per se, or can be delivered via NORS.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

The term “subject” or “veterinary subject” may be used interchangeably and refer to a non-human animal or individual that may benefit from the administration of NORS or gNO produced by NORS. Some non-limiting examples of veterinary subjects can include a bovine, goat, swine, foul, canine, feline, horse, bison, alpaca, llama, sheep, and the like. In one embodiment, the veterinary subject can be a bovine. In another embodiment, the veterinary subject can be a chicken, rooster, duck, goose, pheasant, or other fowl. In another embodiment, the veterinary subject can be a pig or other swine. In another embodiment, the veterinary subject can be a dog. In another embodiment, the veterinary subject can be a cat. In a further embodiment, can be a ferret or a mink. In yet another embodiment, the veterinary subject can be a commercially salable animal. Occurrences of the phrase “in one embodiment,” or “in one aspect,” herein do not necessarily all refer to the same embodiment or aspect.

A “therapeutic” treatment is a treatment administered to a subject who exhibits signs and/or symptoms of a disease or disorder, for the purpose of diminishing or eliminating those signs and/or symptoms. Additionally a “therapeutic” treatment may be a treatment administered to a subject who does not exhibit signs and/or symptoms of a disease or disorder, but who is determined to be at risk for development of a given disease or disorder, for the purpose of preventing or delaying onset of such disease or disorder.

As used herein a “therapeutic agent” refers to an agent that can have a beneficial or positive effect on a subject when administered to the subject in an appropriate or effective amount. In one aspect, NO can be a therapeutic agent.

As used herein, the terms “treat,” “treatment,” or “treating” when used in conjunction with the administration of NORS, including compositions and dosage forms thereof, refers to administration to subjects who are either asymptomatic or symptomatic. In other words, “treat,” “treatment,” or “treating” can be to reduce, ameliorate or eliminate symptoms associated with a condition present in a subject, or can be prophylactic, (i.e. to prevent or reduce the occurrence of the symptoms in a subject). Such prophylactic treatment can also be referred to as prevention of the condition. Further, these terms can encompass metaphylactic acts of administering NORS to bovine in anticipation of an expected outbreak of disease. Moreover, a “treatment outcome” refers to a result obtained at least in part, due to behavior or an act taken with regard to a subject. Treatment outcomes can be expected or unexpected. In one specific aspect, a treatment outcome can be a delay in occurrence or onset of a disease or conditions or the signs or symptoms thereof.

As used herein, the term “metaphylactic” refers to acts of mass medication of a group of subjects as a matter of policy or procedure to minimize or prevent the outbreak of a disease or disorder.

As used herein, an “effective amount” of an agent is an amount sufficient to accomplish a specified task or function desired of the agent. The phrase “therapeutically effective amount,” as used herein, refers to an amount that is sufficient or effective to prevent or treat (delay or prevent the onset of, prevent the progression of, inhibit, decrease or reverse) a disease or disorder in a subject. It is understood that various biological factors may affect the ability of a substance to perform its intended task. Therefore, a “therapeutically effective amount” may be dependent in some instances on such biological factors. Further, while the achievement of therapeutic effects may be measured by veterinarian, or other qualified veterinary personnel using evaluations known in the art, it is recognized that individual variation and response to treatments may make the achievement of therapeutic effects a somewhat subjective decision. The determination of an effective amount or therapeutically effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986).

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

The present disclosure provides an animal intranasal administration device, and associated systems and methods related to a nitric oxide releasing solution (NORS) capable of reducing the presence of a bacteria, virus, or other pathogen in a subject animal. Subject animals can include any suitable animal, such as a mammal. In particular, mammals such as domesticated animals (e.g., bovine, swine, equine, ovine, goat, canine, feline, etc.) are contemplated herein. In one aspect, the present disclosure provides a method and apparatus for treating a subject animal with the delivery of a nitric oxide releasing solution to a treatment site of the animal, such as at least a portion of an upper respiratory tract of the animal. The principles of the present disclosure can be used to treat, prevent, or reduce the incidence of any disease, disorder, or condition where nitric oxide delivery is beneficial. Exemplary diseases, disorders, or conditions, include but are not limited to, respiratory diseases (e.g., BRDc), respiratory infections, wounds, burns, topical infections, inflammatory diseases, and the like.

The present disclosure allows for delivery of nitric oxide to an ambulatory veterinary subject, or to an assembly line of veterinary subjects where the administration protocol for delivery of the nitric oxide releasing solution is accomplished in a short time period. For example, the extended release and delivery of nitric oxide to the treatment site by way of the administered nitric oxide releasing solution allows for the treated subject to remain ambulatory during treatment, or stationary for a very short period of time. Thus, the veterinary subject is not constrained to a nitric oxide delivery device during the entire duration of nitric oxide delivery. Rather, the nitric oxide releasing solution can be administered to the subject over a short duration of treatment, and following administration the nitric oxide releasing solution will continue to deliver an extended release of a therapeutically effective amount of nitric oxide to the subject. The ability for the subject to remain ambulatory during treatment is particularly important in cattle, because cattle or other veterinary subjects can become stressed when they are restrained, such as in a squeeze chute, and stress can exacerbate and increase the incidence of BRDc. The present disclosure provides methods of treatment in any suitable subject, including primates, mammals, cattle, horses, dogs, cats, pigs, sheep, goats, and the like. In one embodiment, the subject is a bovine.

In certain embodiments, the nitric oxide releasing solution is prepared just prior to administration to the subject through the administration of an acidifying or activation agent (e.g., citric acid) to a dormant NORS solution. Alternatively, a sodium nitrite can be administered to a dormant acidified solution. Either mechanism can be selected and used based on a number of performance factors such as most stable shelf life, etc. For example, administration of the acidifying agent to the dormant solution results in the lowering of the pH of the dormant solution, thereby activating the nitric oxide releasing solution to be administered to the treatment site. Importantly, the nitric oxide releasing solution can provide for extended production of nitric oxide, for example, beyond the time required to administer the nitric oxide releasing solution. In one embodiment, the nitric oxide releasing solution produces nitric oxide for a period of between 1 minute and 24 hours. In one embodiment, the nitric oxide releasing solution produces nitric oxide for a period of between 10 and 45 minutes. In one embodiment, the nitric oxide releasing solution produces nitric oxide for at least 15 minutes. In one embodiment, the nitric oxide releasing solution produces nitric oxide for at least 30 minutes. In another embodiment, the nitric oxide releasing solution produces nitric oxide for at least 1 hour. In another embodiment, the nitric oxide releasing solution produces nitric oxide for at least 4 hours. In another embodiment, the nitric oxide releasing solution produces nitric oxide for at least 8 hours. In another embodiment, the nitric oxide releasing solution produces nitric oxide for at least 12 hours. In another embodiment, the nitric oxide releasing solution produces nitric oxide for at least 24 hours. Thus, the administered nitric oxide releasing solution provides for continuous delivery of nitric oxide to the treatment site of the subject. It should be noted that in some embodiments, the treatment site can be at or near the location of NORS administration, for example, the upper respiratory tract. However, in some embodiments, the treatment site (i.e. the location where nitric oxide therapy is desired) can be distal from the location of NORS administration (e.g. the lower respiratory tract).

The nitric oxide releasing solution may be administered to the subject in a variety of forms. The nitric oxide releasing solution may be administered as a liquid, a spray, a vapor, micro-droplets, mist, or any form which provides the release of nitric oxide from the solution, as would be understood by one skilled in the art. In one embodiment, the nitric oxide releasing solution is administered as a spray. In another embodiment, the nitric oxide releasing solution is administered as a vapor. In another embodiment, the nitric oxide is administered as a gas. The amount or dosing volume of administered nitric oxide releasing solution may be varied in order to optimize the duration of nitric oxide production and delivery. In one embodiment, the amount of nitric oxide releasing solution administered to a subject is between about 0.1 mL and 5000 mL. In another embodiment, the amount of nitric oxide releasing solution administered to a subject is between about 10 mL and 1000 mL. In one embodiment, the amount of nitric oxide releasing solution administered to a subject is about 2 mL. In one embodiment, the amount of nitric oxide releasing solution administered to a subject is about 10 mL. In one embodiment, the amount of nitric oxide releasing solution administered to a subject is about 32 mL. In another embodiment, the amount of nitric oxide releasing solution administered to a subject is about 160 mL. These amounts or others may be administered in a single spray or in multiple sprays (e.g. 2, 3, 4, 5, 6, or 8-10 sprays) within a given dosage time, for example within 1 minute, 30 seconds, 10 seconds, 5 seconds, 2 seconds, or any other window deemed suitable or beneficial for administering single or multiple sprays. The nitric oxide releasing solution may be readministered one or more times, as necessary to effectively treat the subject. In one embodiment, the nitric oxide releasing solution is administered once to a subject. In another embodiment, the nitric oxide releasing solution is administered multiple times to a subject, where the nitric oxide releasing solution is readministered substantially after completion of the extended release of nitric oxide gas from the prior dosage administered.

In certain embodiments, nitric oxide releasing solution is directly administered into the upper respiratory tract of the subject. For example, in one embodiment, the nitric oxide releasing solution is sprayed into the upper respiratory tract of the subject. The solution may be administered into the upper respiratory tract of the subject once an hour, once a day, once a week, once every two weeks, once a month, once every two months, once a year, and any and all ranges therebetween as required to treat the subject. In one embodiment, the solution is sprayed once a week. In another embodiment, the solution is sprayed once a week for four consecutive weeks. The nitric oxide releasing solution provides for extended nitric oxide production, thereby providing continuous delivery of therapeutic nitric oxide to the respiratory system of the subject.

The duration of administering the nitric oxide releasing solution to the subject may be varied in order to obtain a desired delivery. In one embodiment, the nitric oxide releasing solution is administered to the subject over a time period of less than 5 seconds. In another embodiment, the nitric oxide releasing solution is administered to the subject over a time period of about 5 seconds. In another embodiment, the nitric oxide releasing solution is administered to the subject over a time period of about 30 seconds. In another embodiment, the nitric oxide releasing solution is administered to the subject over a time period of about 1 minute. In another embodiment, the nitric oxide releasing solution is administered to the subject over a time period of about 2 minutes. In another embodiment, the nitric oxide releasing solution is administered to the subject over a time period of about 10 minutes. In another embodiment, the nitric oxide releasing solution is administered to the subject over a time period of about 30 minutes.

In one aspect, the principles disclosed herein provide for the treatment, prevention, or reduction of incidence of a respiratory disease or disorder in a subject. Exemplary respiratory diseases or disorders that can be treated include, but are not limited to BRDc, porcine respiratory disease complex (PRDc), and the like. In some cases, the respiratory disease or disorder may be caused by a bacterium (e.g., M. haemolytica, H. somni, mycobacteria), fungus, a virus (e.g., Infectious Bovine Rhinotracheitis (IBR), Bovine Parainfluenza-3 (PI-3), and Bovine Respiratory Syncytial Virus (BRSV)), a protozoan, a parasite, and/or an arthropod, including a bacterium that has developed resistance to one or more antibiotics. Treatment of a respiratory disease by way of the present disclosure comprises the delivery of a nitric oxide releasing solution into the upper respiratory tract of the subject to be treated. For example, in certain embodiments, the nitric oxide releasing solution may be injected, sprayed, inhaled, or instilled into the respiratory tract of the subject. The nitric oxide releasing solution may be administered to the respiratory tract of the subject via the nasal cavity or oral cavity of the subject. In one embodiment, the nitric oxide releasing solution is sprayed into the upper respiratory tract of the subject. In one embodiment, the solution is administered to the subject intranasally. In one embodiment, the solution is administered to the sinuses. The nitric oxide releasing solution provides for extended nitric oxide production, thereby providing continuous delivery of therapeutic nitric oxide to the respiratory tract of the subject.

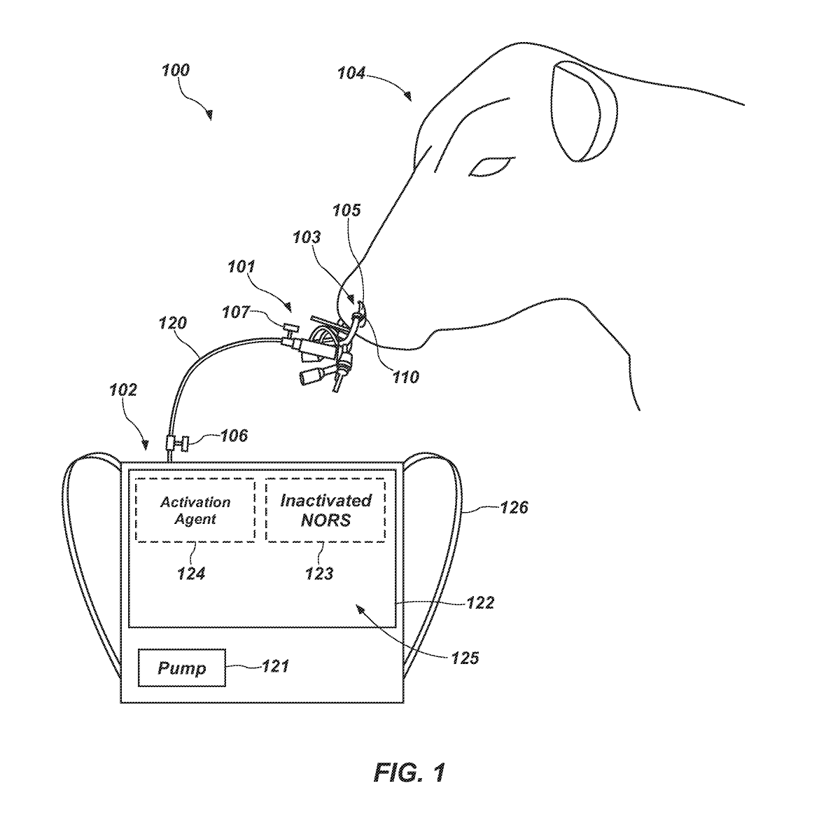

With reference to FIG. 1, illustrated is an animal intranasal administration system 100 in accordance with an example of the present disclosure. The system 100 can include an animal intranasal administration device 101 that can be used for administering a fluid (e.g., nitric oxide releasing solution) to a nostril 103 of an animal 104. The system 100 can also include a fluid source 102 to provide the fluid to the intranasal administration device 101. In one aspect, the fluid provided by the fluid source 102 and/or administered by the device 101 to the animal 104 can be in a liquid or gas state. In some embodiments, the liquid may be prepared to have a desired viscosity.

The intranasal administration device 101 can include a nasal passage nozzle 110 for each nostril configured to receive fluid from the fluid source 102 fluidly coupled to the nasal passage nozzles, such as via a fluid conduit 120. The intranasal administration device 101 can also include a biasing mechanism to bias the nozzles toward a septum 105 of the animal 104, such that the device is secured in place about the septum during administration of the fluid into nasal passages of the animal. The biasing action of each nozzle toward the septum allows the nozzles or other parts of the device to effective pinch the septum as they are on opposite sides thereof. The device can then be held in place as it pinches the septum. The animal intranasal administration system 100 can also include a pump 121 operable to deliver fluid from the fluid source 102 to the nasal passage nozzles 110. The pump 121 can be a motorized pump powered by electricity and/or a hand-operated pump. Any pump that is sufficient to deliver NORS in a volume and at a velocity that provides effective NO treatment can be used. In one example, NORS can be delivered at a velocity sufficient to ensure delivery of NORS liquid to the pharyngeal tonsillar material in the upper airway. Other deliver parameters and characteristics, such as volume, delivery time and variation can be selected and controlled in order to achieve a specific result, such as placing a specific volume of NORS at a specific physical location within a subject can be used, for example a set volume can be delivered with varying pressure, or a set time with a fixed pressure can be used to achieve a desired volume.

In one example, a hand-operated pump (e.g., a trigger operated vacuum hand pump) can be coupled to the fluid conduit 120 “inline” to deliver the fluid to the device 101 without the use of electricity. In one aspect, the fluid source can be portable by a user while in use. In some embodiments, the system 100 can include one or more carrying straps 126 coupleable to the fluid source 102 (e.g., directly coupled or coupled via a backpack or other carrying case) to facilitate portability by the user. Thus, in certain embodiments, the system 100 can be portable and powered entirely by the user. In alternative embodiments, the fluid source can be substantially stationary and in some cases can be attached to a post or other fixture. This embodiment can be advantageous when treating a large number of subjects as it allows a large volume of nitric oxide releasing solution to be utilized (i.e. from a large container).

The system 100 can include one or more valves associated with the fluid source 112, fluid conduit 120, and/or the device 101 to control the flow of fluid to the nasal passage nozzles 110, such as to control a fluid dosage to the animal 104. For example, a valve 106 can be located at or near the fluid source 102 and a valve 107 can be located at or near the device 101, although a valve may be disposed in any suitable location. In one aspect, a valve can be associated with one or both of the nasal passage nozzles 110 to control the flow of fluid to a specific nozzle. Any other mechanism for metering out a specific volume or dose of nitric oxide releasing solution for administration to the subject can also be used, including simply the amount of time over which the solution is administered (i.e. administration period) in combination with flow rate, etc.

In some embodiments, the fluid source 102 can comprise inactivated nitric oxide releasing solution 123, an activation agent 124, activated nitric oxide releasing solution, and/or nitric oxide gas. The activation agent 124 can be configured to activate the inactivated nitric oxide releasing solution 123 upon mixing. In one aspect, the activation agent 124 can be maintained separate from the inactivated nitric oxide releasing solution 123. The activation agent 124 can be in any suitable form, such as a solid (e.g., a powder, a tablet, and a capsule), a liquid (e.g., a solution), a gas, etc. The fluid source 102 can also comprise one or more containers 122 or reservoirs for the inactivated nitric oxide releasing solution 123, the activation agent 124, activated nitric oxide releasing solution, and/or nitric oxide gas. In general, the activation agent 124 and the inactivated nitric oxide releasing solution 123 can be at least partially mixed in a mixing chamber 125, which can be within the container 122. Thus, in one aspect, the inactivated nitric oxide releasing solution 123 can be activated within the container 122 and dispensed or delivered to the device 101 to be administered to the animal 104. The pump 121 can convey activated nitric oxide releasing solution from the fluid source 102 to the device 101. Alternatively, activated nitric oxide releasing solution can be conveyed from the fluid source 102 to the device 101 by pressure in the container 122 due to the production of nitric oxide gas resulting from activation of the nitric oxide releasing solution. In other words, an increase in gas pressure in the container 122, due to the formation of nitric oxide, can cause activated nitric oxide releasing solution to move from the container 122 to the device 101 via the fluid conduit 120 for delivery to the animal. In such embodiments, pump 121 may not be needed, or can be utilized if the pressure inside the container 122, becomes insufficient to continue dispensing the nitric oxide releasing solution at the desired rate/volume. In an alternative embodiment as described more fully below, a pump, either electric or manually operated, can be used to create pressure within the container and facilitate administration of the nitric oxide releasing solution.

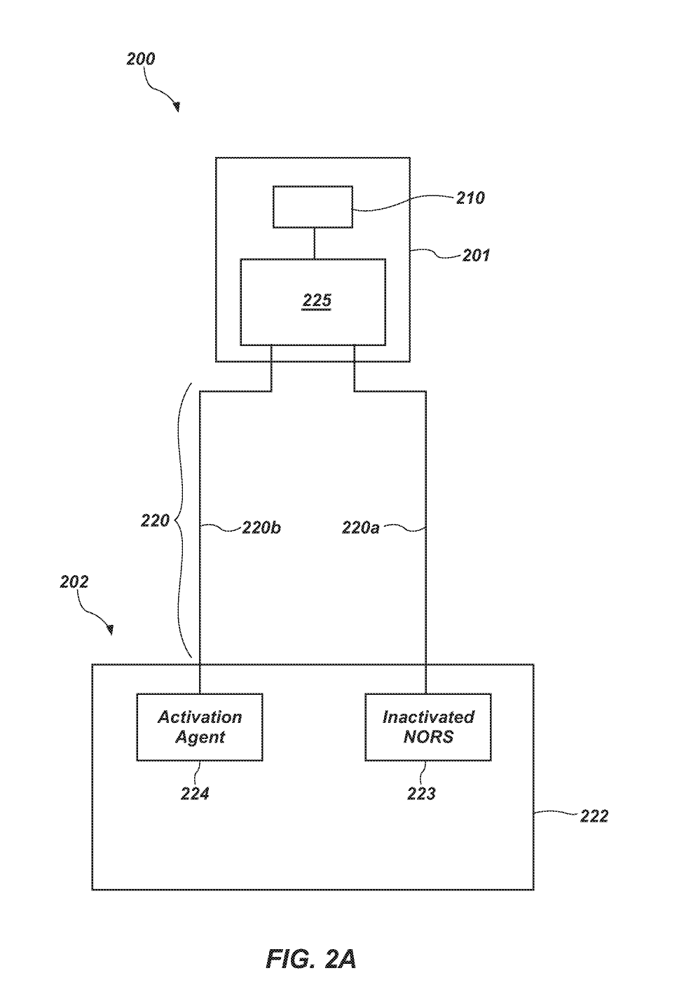

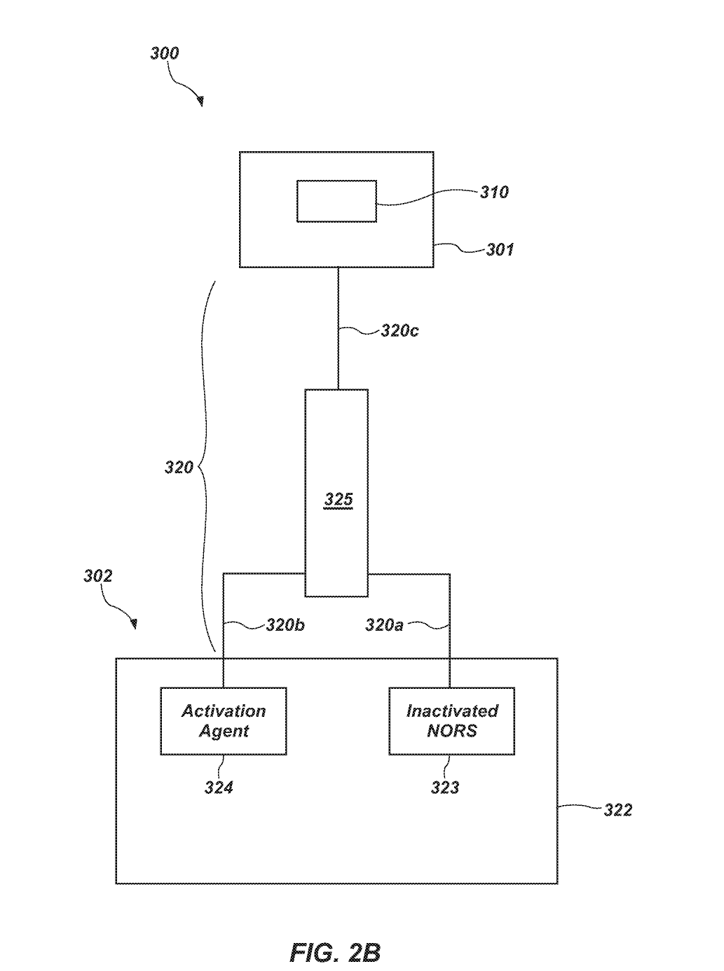

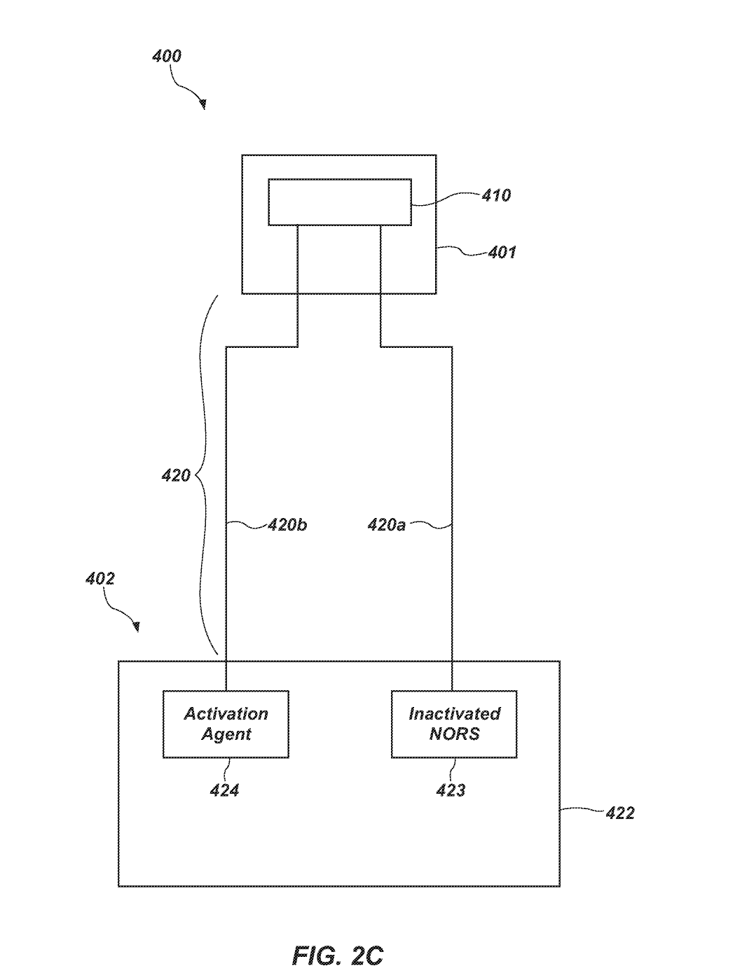

In another aspect, illustrated in FIGS. 2A-2C, an activation agent and inactivated nitric oxide releasing solution can be at least partially mixed in a mixing chamber external to a container, such as the container 122 of FIG. 1. For example, as shown in FIG. 2A, an intranasal administration system 200 can include a fluid source 202 fluidly coupled to an intranasal administration device 201 (e.g., to nasal passage nozzles 210) via a conduit 220, which includes a conduit 220a associated with inactivated nitric oxide releasing solution 223 and a conduit 220b associated with an activation agent 224, each of which can be disposed in separate containers. The conduits 220a, 220b can combine prior to the nasal passage nozzles 210, such as in a mixing chamber 225 within the intranasal administration device 201, such that mixing of the inactivated nitric oxide releasing solution 223 and the activation agent 224 occurs between the fluid source 202 and the nasal passage nozzles 210. Thus the nitric oxide releasing solution can be activated, or in other words, the activated solution can be formed, during delivery or administration of the nitric oxide releasing solution to a subject.

In another example, shown in FIG. 2B, an intranasal administration system 300 can include a fluid source 302 fluidly coupled to an intranasal administration device 301 (e.g., to nasal passage nozzles 310) via a conduit 320, which includes a conduit 320a associated with inactivated nitric oxide releasing solution 323 and a conduit 320b associated with an activation agent 324, each of which can be disposed in separate containers. The conduits 320a, 320b can combine prior to the nasal passage nozzles 310, such as in a mixing chamber 325 external to the fluid source 302 and the intranasal administration device 301, such that mixing of the inactivated nitric oxide releasing solution 323 and the activation agent 324 occurs between the fluid source 302 and the nasal passage nozzles 310. In one aspect, the mixing chamber 325 can comprise at least a portion of the conduit 320 such that mixing of the inactivated nitric oxide releasing solution 323 and the activation agent 324 takes place “in-line” to the intranasal administration device 301. Accordingly, the mixing chamber 325 can comprise any suitable structure, such as tubing, that can be disposed between the fluid chamber 302 and the intranasal administration device 301 and serve to mix the inactivated nitric oxide releasing solution 323 and the activation agent 324. The mixing chamber 325 can form an integral part of tubing that forms the conduit 320 or the mixing chamber 325 can be a separate component coupled to tubing to form a portion of the conduit 320. Activated nitric oxide releasing solution can be conveyed to the intranasal administration device 301 from the mixing chamber 325 via conduit 320c.

In yet another example, shown in FIG. 2C, an intranasal administration system 400 can include a fluid source 402 fluidly coupled to an intranasal administration device 401 (e.g., to nasal passage nozzles 410) via a conduit 420, which includes a conduit 420a associated with inactivated nitric oxide releasing solution 423 and a conduit 420b associated with activation agent 424, each of which can be disposed in separate containers. The conduits 420a, 420b can combine at the nasal passage nozzles 410, which can form a mixing chamber, such that mixing of the inactivated nitric oxide releasing solution 423 and the activation agent 424 occurs at the nasal passage nozzles 410. Accordingly, the nasal passage nozzles 410 can comprise any suitable structure that can serve to accommodate the introduction of solution from multiple conduits and mix the inactivated nitric oxide releasing solution 423 and the activation agent 424. Thus, the conduits 420a, 420b can remain separate from the fluid source 402 to the nasal passage nozzles 410 such that mixing of the inactivated nitric oxide releasing solution and the activation agent occurs at an animal engaged by the intranasal administration device 401. In other words, the nitric oxide releasing solution is activated or formed in-vivo at the administration site, or after being dispensed from the nozzle.

In one aspect, each nasal passage nozzle can receive either an activation solution or inactivated nitric oxide releasing solution, such that each is administered to the animal separately. Thus, the activation solution and the inactivated nitric oxide releasing solution can mix after being dispensed from the intranasal administration device at or inside the animal, such as inside a nasal passage, to activate the nitric oxide releasing solution. In some embodiments, each nozzle may have separate openings and supporting fluidic connections to the respective sources of activation agent and nitrite solution (i.e. inactivated NORS). In this way, solution from each source can be brought to the nozzle separately, yet simultaneously for delivery to a subject concurrently. A nozzle can have a single opening and the solutions can be alternately administered, for example, a spray of inactivated NORS (i.e. nitrite solution, citric acid) followed by a spray of activator solution (e.g. citric acid, ascorbic acid, nitrite solution, etc.).

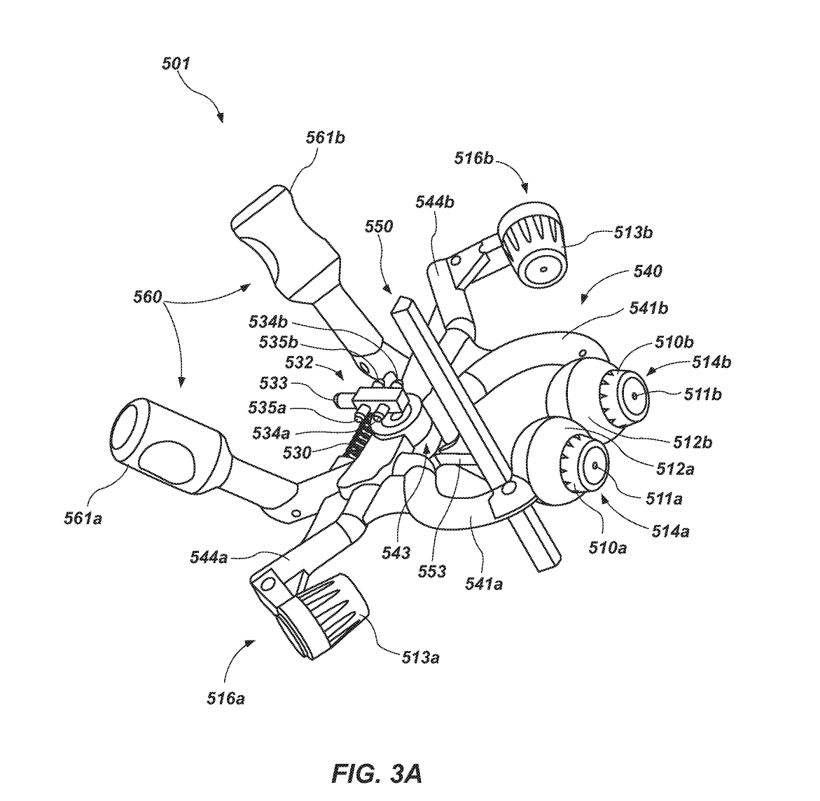

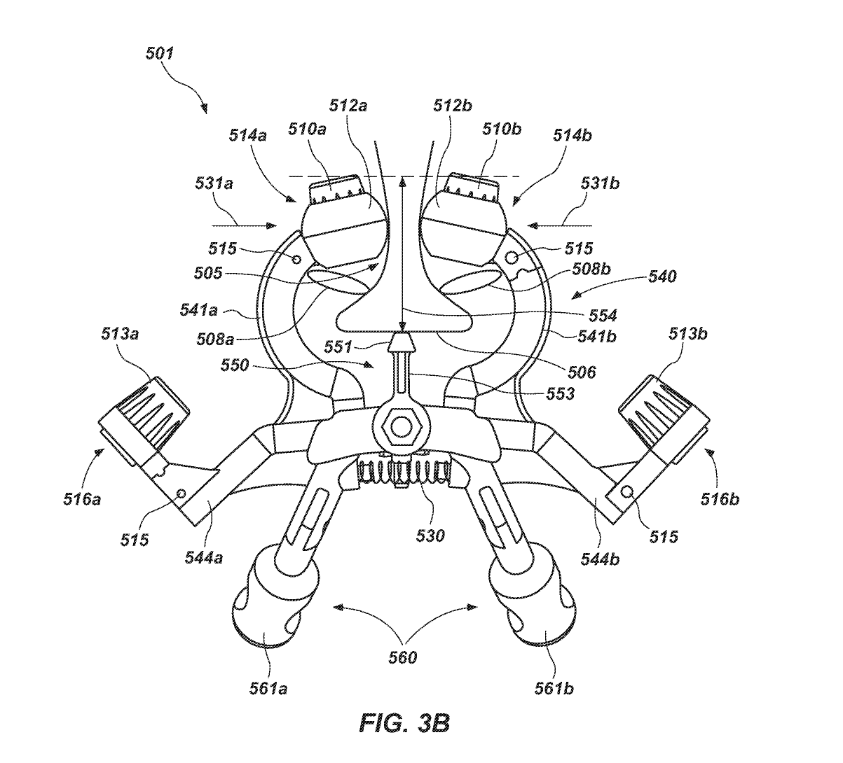

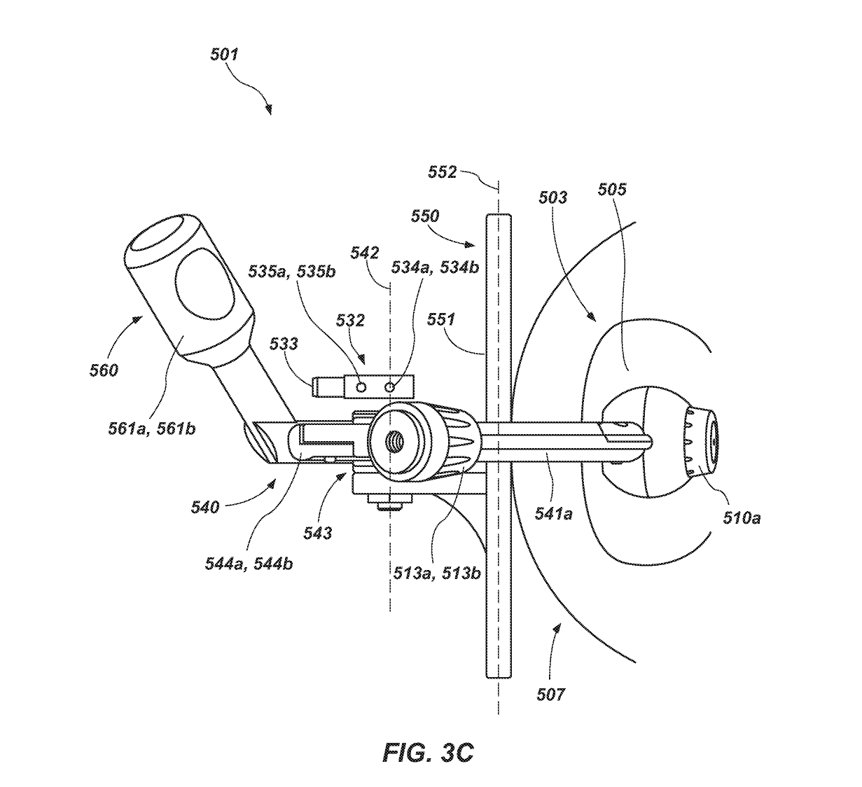

FIGS. 3A-3C illustrate an animal intranasal administration device 501 in accordance with an example of the present disclosure. The intranasal administration device 501 can include a nasal passage nozzle 510a, 510b for each nostril 503 (FIG. 3C) configured to receive fluid from a fluid source, as described hereinabove. The intranasal administration device 501 can also include a biasing mechanism 530 to bias the nozzles 510a, 510b toward a septum 505 (FIGS. 3B and 3C) of an animal, such that the device 501 is secured in place about the septum 505 during administration of the fluid into nasal passages of the animal.

In one aspect, the intranasal administration device 501 can include a support member 540 having support member portions 541a, 541b coupled to, and in support of, the nasal passage nozzles 510a, 510b, respectively. The support member portions 541a, 541b can be movable relative to one another (i.e., rotatably coupled to one another at pivot coupling 543) to secure the nasal passage nozzles 510a, 510b at least partially within the nostrils 503 of the animal about the septum 505 and such that fluid is directed into nasal passages of the animal. Thus, the nasal passage nozzles 510a, 510b can be oriented to align nozzle openings 511a, 511b with nasal passages when the device 501 is engaged with the septum 505 of the animal to provide for delivery of fluid to deep nasal passages.

In one aspect, the nasal passage nozzles 510a, 510b can be configured to direct fluid into the nasal passages past nasal folds 508a, 508b which may exist in the animal, as represented in FIG. 3B. For example, a bovine may have an alar fold, a basal fold, and a straight fold. Thus, the nasal passage nozzles 510a, 510b can be configured to direct fluid into the nasal passages past one or more of such folds to deliver the fluid to deep nasal passages. In one example, the nasal passage nozzles 510a, 510b can be configured to extend or penetrate into the nostrils beyond one or more nasal folds 508a, 508b, as illustrated in FIG. 3B to reach as far as the nasopharyngeal tonsillar material. In another example, the nasal passage nozzles 510a, 510b can be located and oriented to direct the fluid past one or more nasal folds without extending or penetrating into the nostrils beyond one or more of the nasal folds. In short, any configuration required to effectively administer nitric oxide releasing solution into the nasal passages, or any other desired or specified location in the respiratory tract of any subject in a manner sufficient to allow the subject to receive effective nitric oxide therapy, given the subject's specific anatomy, can be used.

In one aspect, the support member portions 541a, 541b can be movable relative to one another by the biasing mechanism 530 to bias the nasal passage nozzles 510a, 510b toward a secured position about the septum 50 in direction 531a, 531b. For example, the biasing mechanism 530 can comprise a spring acting on the support member portions 541a, 541b to bias the support member portions 541a, 541b toward the secured position about the septum 505. The biasing mechanism 530 can therefore cause the nasal passage nozzles 510a, 510b to pinch the septum 505 therebetween so that the nozzles 510a, 510b are held in place in the nostrils 503. While illustrated as a spring, it is to be understood that the biasing mechanism 530 can be any device, part, or mechanism that is sufficient to provide the desired biasing action. Moreover, the biasing mechanism 530 can be located anywhere on the device 501 that is adequate to provide the desired biasing action. In one aspect, biasing or spring strength can be adjustable as desired to secure the device 501 to the animal without causing undue pain to the animal. In one aspect, the support member 540 can be configured to provide clearance about a tip 506 of the septum 505. For example, the support member portions 541a, 541b can comprise arcuate configurations to provide clearance about the tip 506 of the septum 505, as illustrated in FIG. 3B.

The intranasal administration device 501 can include a septum interface portion 512a, 512b associated with the nasal passage nozzles 510a, 510b, respectively, to interface with the septum 505 and position the nasal passage nozzles to facilitate directing fluid deep into the nasal passages of the animal. For example, the septum interface portion 512a, 512b can serve to space or position the nasal passage nozzles 510a, 510b and openings 511a, 511b at a sufficient distance from the septum 505 to facilitate and maintain dispersal or spray pattern coverage into the nasal passages without interference from the septum 505.

The intranasal administration device 501 can also include a positioning member 550 configured to contact the tip 506 of the septum 505 to facilitate and maintain proper positioning and/or orientation of the nasal passage nozzles 510a, 510b within the nostrils 503 of the subject so that the nasal passage nozzles 510a, 510b direct fluid in a direction substantially aligned with the nasal passage openings of the animal. For example, the positioning member 550 can be configured to position the nasal passage nozzles 510a, 510b such that the openings 511a, 511b are at a distance 554 from the tip 506 of the septum 505 to properly position the nasal passage nozzles 510a, 510b at a suitable distance relative to the nasal passage openings. In one aspect, the positioning member 550 can comprise an elongated portion 551 having a longitudinal axis 552 that is substantially parallel to an axis 542 of rotation for movement of the support member portions 541a, 541b relative to one another. For example, the positioning member 550 can have a “T” configuration where a base portion 553 supports the elongated portion 551. The base portion 553 can be coupled to the support member 540, such as to one or both of the support member portions 541a, 541b, at the pivot coupling 543 of the support member portions 541a, 541b. The elongated portion 551 can be configured to contact a muzzle 507 of the animal to prevent or minimize sagging or downward rotation of the device 501 during use, thereby facilitating proper alignment of the nasal passage nozzles 510a, 510b.

The intranasal administration device 501 can include a user interface 560 coupled to the support member 540 to facilitate movement of the support member portions 541a, 541b relative to one another by a user. For example, the user interface 560 can include user interface portions 561a, 561b, such as handles, coupled to the support member portions 541a, 541b, respectively, to facilitate movement of the nasal passage nozzles 510a, 510b by a user in a direction opposite the biasing direction 531a, 531b, such as by squeezing the user interface portions 561a, 561b toward one another.

In one aspect, the intranasal administration device 501 can include one or more nostril nozzles 513a, 513b configured to direct fluid onto the nostrils 503 of the subject. In a particular aspect, the nostril nozzles 513a, 513b can be configured to direct fluid onto the anterior nostrils. The nostril nozzles 513a, 513b can be coupled to the support member 540. For example, the support member 540 can comprise lateral extension portions 544a, 544b to position the nostril nozzles 513a, 513b, respectively. In one aspect, the lateral extension portions 544a, 544b can be coupled to, and extend from, the support member portions 541a, 541b, respectively. In another aspect, the intranasal administration device 501 can include one or more muzzle nozzles (not shown in these figures) configured to direct fluid onto the muzzle 507 of the animal. A muzzle nozzle can be supported by one or more of the support member portions 541a, 541b and/or the lateral extension portions 544a, 544b. As such, delivery of the nitric oxide releasing solution can be made to both the nasal passages and the nares simultaneously, or at the very least, using a single device.

Although the intranasal administration device 501 is shown with four total nozzles, it should be recognized that an intranasal administration device in accordance with the present disclosure can include any suitable number of nozzles, which can have an appropriate dispersal or spray pattern directed at an appropriate angle to any suitable area of an animal's muzzle, nares, nostrils, nasal passage, etc. In other words, nozzle dispersal or spray patterns can be specifically suited for a particular area (i.e., the nasal passages, nostrils, muzzle, etc.) and can be oriented at any suitable angle to direct fluid onto or into the area. In one aspect, one nozzle can be configured to direct fluid onto multiple areas. For example, the nostril nozzles 513a, 513b can be configured to disperse or spray fluid on the nares and the muzzle. Thus, the nozzles of an intranasal administration device in accordance with the present disclosure can be configured to have various dispersal or spray patterns to cover nasal passages and entry surfaces into the nasal passages. Nozzles used with the device 501 may therefore initiate any spray pattern known in the art suitable for a given purpose or dispersing target region.

In one aspect, the intranasal administration device 501 can include a fluid distribution manifold 532 fluidly coupled to the nozzles of the device 501. For clarity, external fluid couplings or conduits, such as tubing or hoses, have been omitted. The fluid distribution manifold 532 can have an inlet port 533 to receive fluid from a fluid source and outlet ports 534a, 534b, 535a, 535b to distribute fluid to the various nozzles of the device 501. For example, outlet ports 534a, 534b can be fluidly coupled to the nasal passage nozzles 510a, 510b, respectively, and outlet ports 535a, 535b can be fluidly coupled to the nostril nozzles 513a, 513b, respectively. Thus, each of the nasal passage nozzles 510a, 510b and the nostril nozzles 513a, 513b can be configured to couple with a conduit to receive fluid from a fluid source. Although the fluid distribution manifold 532 is shown separate from other structural components of the device 501, such as the support member 540 or the positioning member 550, it should be recognized that a fluid distribution manifold can be coupled to or integrally formed with any structural portion of the device 501, such as one or more portions of the support member 540 and/or the positioning member 550. In one aspect, the fluid manifold 532 can include at least two inlet ports and a mixing chamber, as discussed above, such that mixing of inactivated nitric oxide releasing solution and activation agent occurs between a fluid source and the nasal passage nozzles 510a, 510b. In another aspect, the fluid distribution manifold 532 can include one or more valves to control fluid flow one or more nozzles of the device 501.

In one aspect, the support member 540 can have internal fluid conduits defined by one or more openings or passageways through the support member 540. For example, one or more of the support member portions 541a, 541b can include at least a portion of a fluid conduit to direct fluid to the respective nasal passage nozzle 510a, 510b from the fluid source. Similarly, one or more of the lateral extension portions 544a, 544b can include at least a portion of a fluid conduit to direct fluid to the respective nostril nozzle 513a, 513b from the fluid source. Thus, such internal fluid conduits can receive fluid directly from the fluid source or after distribution from the fluid distribution manifold 532.

In one aspect, the intranasal administration device 501 can be constructed to facilitate interchangeability of parts. For example, the support member portions 541a, 541b can be configured to removably couple with nozzle or spray heads 514a, 514b, such as with fasteners 515. Similarly, the lateral extension portions 544a, 544b can be configured to removably couple with nozzle or spray heads 516a, 516b, such as with fasteners 515. In addition, the support member portions 541a, 541b can be configured to removably couple with the user interface portions 561a, 561b. Furthermore, the biasing member or spring 530 can be removably coupled to the support member 540. Thus, nozzles, springs, handles, positioning members, etc. can be interchangeable and replaced as desired to accommodate different animal species and/or animals of a different size. Thus, the device 501 can be configured and customized for the anatomy of a cow of a given age. In one aspect, the intranasal administration device 501 can be disassembled to facilitate cleaning and/or servicing of the various parts or components of the device.

In one aspect, the nozzle or spray heads 514a, 514b can include or incorporate the nasal passage nozzles 510a, 510b as well and the septum interface portions 512a, 512b, respectively. As illustrated in FIGS. 3A-3C, the spray heads 514a, 514b can have a spherical or ball configuration that provides a curved interface surface for the septum interface portions 512a, 512b for contacting the septum 505. Such a spherically curved surface can accommodate various septum thicknesses and maintain a consistent interface with the septum 505. The spherical surface can have a diameter configured to provide adequate surface area for effective “clamping” (i.e. pinching) contact with the septum without providing excessive pressure to the contact area of the septum such that the device 501 is uncomfortable for the animal. The diameter of the spherical surface can also contribute to providing adequate space for the nasal passage nozzles 510a, 510b from the septum to provide and maintain a suitable dispersal or spray pattern.

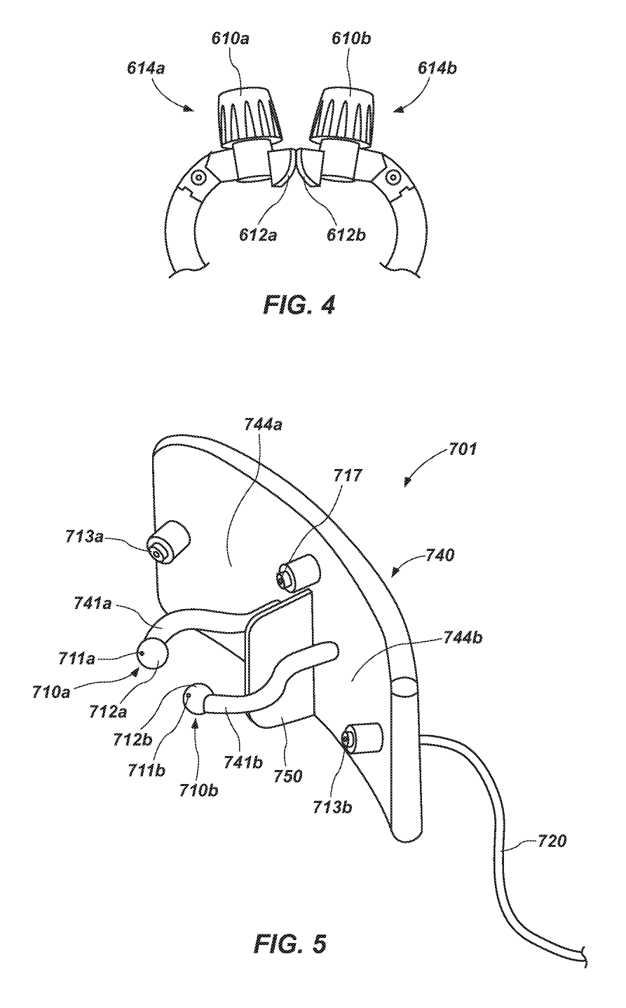

FIG. 4 illustrates nozzle or spray heads 614a, 614b in accordance with another example of the present disclosure. As with the spray heads 514a, 514b of FIGS. 3A-3C discussed above, the spray heads 614a, 614b can include or incorporate nasal passage nozzles 610a, 610b as well as septum interface portions 612a, 612b, respectively. In this case, the spray heads 614a, 614b have a fan configuration with an arcuate surface for the septum interface portions 612a, 612b for contacting a septum. Such an arcuate curved surface can accommodate various septum thicknesses and may be useful when a higher contact pressure is desired, due to the relatively small contact area that can be provided by this configuration. The size of the arcuate surface can also contribute to providing adequate space for the nasal passage nozzles 610a, 610b from a septum to provide and maintain a suitable dispersal or spray pattern.

FIG. 5 illustrates an animal intranasal administration device 701 in accordance with another example of the present disclosure. The intranasal administration device 701 can include a nasal passage nozzle 710a, 710b for each nostril configured to receive fluid from a fluid source, as described hereinabove. In one aspect, the intranasal administration device 701 can include a support member 740 having support member portions 741a, 741b coupled to, and in support of, the nasal passage nozzles 710a, 710b, respectively. In one aspect, the support member 740 can be resiliently flexible or include resiliently flexible components. Thus, in a particular aspect, one or both of the support member portions 741a, 741b can be resiliently flexible and therefore movable relative to one another to secure the nasal passage nozzles 710a, 710b at least partially within the nostrils of an animal about a septum and such that fluid is directed into nasal passages of the animal. The resilient flexibility of the support member portions 741a, 741b can provide a biasing mechanism to bias the nozzles 710a, 710b toward a septum of an animal, such that the device 701 is secured in place about the septum during administration of the fluid into nasal passages of the animal. Thus, the resilient flexibility of the support member portions 741a, 741b can bias the nasal passage nozzles 710a, 710b toward a secured position about the septum 70 in direction 731a, 731b. The nasal passage nozzles 710a, 710b can be oriented to align nozzle openings 711a, 711b with nasal passages when the device 701 is engaged with the septum of the animal to provide for delivery of fluid to deep nasal passages.

The intranasal administration device 701 can also include a septum interface portion 712a, 712b associated with the nasal passage nozzles 710a, 710b, respectively, to interface with the septum and position the nasal passage nozzles to facilitate directing fluid deep into the nasal passages of the animal. For example, the septum interface portion 712a, 712b can serve to space or position the nasal passage nozzles 710a, 710b and openings 711a, 711b away from the septum to facilitate and maintain dispersal or spray pattern coverage into the nasal passages without interference from the septum. The septum interface portions 712a, 712b are illustrated with a spherical configuration, although any suitable configuration may be utilized.

The intranasal administration device 701 can further include a positioning member 750 configured to contact a tip of the septum to facilitate and maintain proper positioning and/or orientation of the nasal passage nozzles 710a, 710b within the nostrils of the animal so that the nasal passage nozzles 710a, 710b direct fluid in a direction substantially aligned with the nasal passage openings of the animal. For example, the positioning member 750 can be configured to position the nasal passage nozzles 710a, 710b such that the openings 711a, 711b are at a distance from the tip of the septum to properly position the nasal passage nozzles 710a, 710b at a suitable distance relative to the nasal passage openings. In one aspect, the positioning member 750 can be coupled to the support member 740, such as between the support member portions 741a, 741b. The positioning member 750 can be configured to contact a muzzle of the animal when the device 701 is engaged with the animal to prevent or minimize sagging or downward rotation of the device 701 during use, thereby facilitating proper alignment of the nasal passage nozzles 710a, 710b.

In one aspect, the intranasal administration device 701 can include one or more nostril nozzles 713a, 713b configured to direct fluid onto the nostrils of the animal. In particular, the nostril nozzles 713a, 713b can be configured to direct fluid onto the anterior nostrils. In one aspect, the nostril nozzles 713a, 713b can be coupled to the support member 740. For example, the support member 740 can comprise lateral extension portions 744a, 744b to position the nostril nozzles 713a, 713b, respectively. In another aspect, the intranasal administration device 701 can include one or more muzzle nozzles 717 configured to direct fluid onto a muzzle of the animal. The muzzle nozzle 717 can be coupled to the support member 740 at any suitable location.

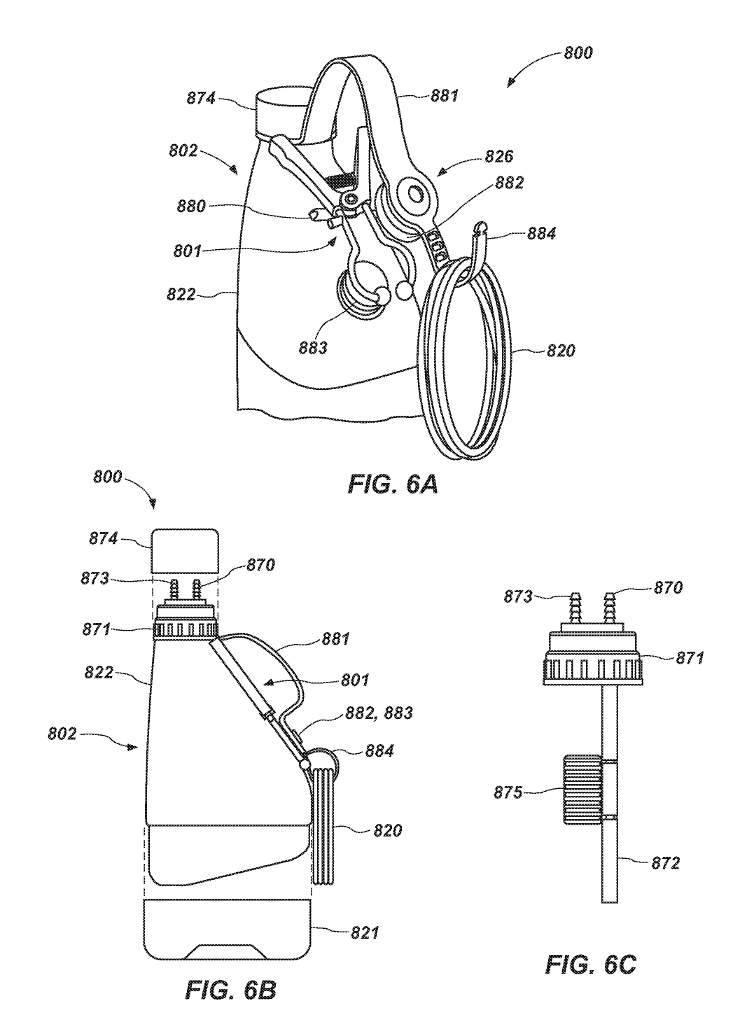

FIGS. 6A-6C illustrate aspects of an animal intranasal administration system 800 in accordance with another example of the present disclosure. The system 800 can include an animal intranasal administration device 801 of any suitable configuration described hereinabove for administering a fluid to a nostril of an animal. The system 800 can also include a fluid source 802 to provide the fluid to the intranasal administration device 801, such as via a fluid conduit 820. The fluid source 802 can comprise inactivated nitric oxide releasing solution, an activation agent, activated nitric oxide releasing solution, and/or nitric oxide gas.

In one aspect, the fluid source 802 can comprise a container 822 or a reservoir with inactivated nitric oxide releasing solution disposed therein. The container 822 may be of any desired size and shape. In one aspect, the container 822 can be suitable for holding multiple doses or application volumes of nitric oxide releasing solution without requiring a refill. The fluid source 802 can also have a fluid outlet port 870, which can be configured to couple with the fluid conduit 820 for delivering the fluid to the device 801. The fluid outlet port 870 can be associated with a cap 871 (as shown) or with the container 822. A sump conduit 872 can be fluidly coupled to the fluid outlet port 870 to deliver fluid to the fluid outlet port 870. The sump conduit 872 will typically extend to a bottom of the container 822 to facilitate evacuating substantially all the fluid from the container 822. The sump conduit 872 can be associated with the cap 871 (as shown) and/or with the container 822 (e.g., molded into a side of the container 822). The fluid source 802 can also include a gas port 873 to allow a gas into the container 822 during use of the system 800. For example, a pump 821 can be a gas pump and can be fluidly coupled to the gas port 873 by a conduit to provide pressurized gas (e.g., air or other suitable gas) to the container 822 such that “head space pressure” in the container 822 causes the fluid to exit the container 822 via the sump conduit 872 and fluid outlet port 870 for delivery to the device 801 through the fluid conduit 820. The gas port 873 can be associated with the cap 871 (as shown) or with the container 822. The gas port 873 will typically be located above a level of the inactivated nitric oxide releasing solution in the container 822. In one aspect, the container 822 can be pressurized to about 50 psig during operation (with about 30 psig being typical), although the system can be configured to operate at any suitable pressure. In one aspect, the pump 821 can provide a pressure to deliver a specific spray volume onto the muzzle and into the nares and nasal passages of an animal. In one aspect, a pressure gage or sensor (i.e., as part of the pump 821) can monitor pressure in the container 822 and/or the fluid conduit 820 to determine whether a nozzle has been clogged.

In one aspect, the pump 821 can be a liquid pump and can operate to pump liquid fluid out of the container 822 without creating head space pressure in the container 822. The pump 821 can be a gas pump and/or a liquid pump of any suitable configuration. In one aspect, the pump 821 can be a motorized pump powered by electricity and/or a hand-operated pump. A cover 874 can be provided for the cap 871 to protect the fluid outlet port 870 and the gas port 873 when not in use. Components of the system can be constructed with metals, plastics, and other polymers compatible with the activation agent (e.g., citric acid, sodium nitrite), nitric oxide releasing solution, and nitric oxide.

In one aspect, the fluid source 802 can include an activation agent maintained separate from the inactivated nitric oxide releasing solution. The activation agent can be configured to activate the inactivated nitric oxide releasing solution upon mixing. Once mixed, the production of nitric oxide in the solution can create a head space pressure sufficient to deliver fluid from the container 822 to the device 801. Thus, fluid can dispense automatically from the device 801 upon mixing the activation agent and the inactivated nitric oxide releasing solution utilizing a gas pressure resulting from the activation of the nitric oxide releasing solution.

The activation agent can be in any suitable form, such as a solid (e.g., a powder, a tablet, a capsule, etc.), a liquid (e.g., a solution), a gas, etc. In one aspect, an activation agent in solid form can be in a dissolvable pouch and/or supported by a cage 875, which can be configured to be disposed within the container 822 below the level of the inactivated nitric oxide releasing solution to ensure contact or mixing with the inactivated nitric oxide releasing solution. The cage 875 can include one or more openings to facilitate mixing of the activation agent and the inactivated nitric oxide releasing solution. Thus, when the activation agent is submerged in the inactivated nitric oxide releasing solution the activation agent will dissolve producing nitric oxide in the solution. The cage 875 can be coupled to the sump conduit 872 (as shown) and supported within the container above a bottom of the container 822 or simply dropped into the container 822. In one aspect, the cage 875 can be coupled to a rod or tube having an end that is located proximate an opening of the container 822. Coupling the cage 875 to the sump conduit 872 or a rod or tube can simplify retrieval of the cage 875.

In one aspect, the animal intranasal administration system 800 can be provided as a kit. For example, the container 822 can have a device coupling feature 880 to couple with and support the device 801. The container 822 can also have a handle 881. The handle 881 can have a free end 826 that can couple to a body of the container 822 via coupling features 882, 883. The coupling features 882, 883 can be configured to further capture and secure the device 801 to the container 822. A fluid conduit coupling feature 884 can extend from the free end 826 of the handle 881 to capture and secure the fluid conduit 820 to the container 822. In addition, the pump 821 can be configured to removably couple with a bottom of the container 822. If the pump 821 includes electrical components, a battery pack may be included. The cover 874 can cover the cap 871 and/or an opening of the container 822 when not in use.

In use of the system 800, an animal can arrive in a holding chute and a user can engage the intranasal administration device 801 with the animal's nostril, as described hereinabove. Because the device 801 is secured to the animal, the user can administer fluid to the animal “hands free.” The fluid source 802 can be supported by a post of the holding chute and can hold a volume (e.g., 5 gallons) of premixed nitric oxide releasing solution in its dormant state. Once the activation agent and the inactive nitric oxide releasing solution are mixed, nitric oxide gas is produced in the solution in the container 822. The activated nitric oxide releasing solution is then conveyed from the fluid source to the device 801 and dispensed or sprayed onto the treatment site or area, such as into the animal's nostrils. For example, the activated solution may be sprayed into the nostrils of the cattle in brief, measured bursts. In one aspect, the animal can receive one spray of about 8 mL into each nostril, twice, for a total of about 32 mL before being released. The duration of treatment administration can be between about 3-5 seconds. At the user's convenience the device 801 can be released or disengaged from the animal. The activated solution now lining the nasal passages of the animal can continue to release nitric oxide gas for up to 30 minutes or longer.

### Examples

The following examples pertain to further embodiments.

In one example, an animal intranasal administration device can comprise a nasal passage nozzle for a nostril configured to receive fluid from a fluid source; a support structure opposing the nasal passage nozzle; and a biasing mechanism to bias the nasal passage nozzle and the support structure toward a septum such that the device is secured in place about the septum during administration of the fluid into a nasal passage.

In on example, the support structure comprises a second nasal passage nozzle.

In one example, the biasing mechanism comprises a spring to bias the nozzles toward the septum.

In one example, the animal intranasal administration device can further comprise a support member having a first support member portion and a second support member portion each in support of a nozzle, wherein the first support member portion and the second support member portion are movable relative to one another by the biasing mechanism.

In one example, the biasing mechanism comprises a spring acting on the first support member portion and the second support member portion.

In one example, the biasing mechanism comprises resilient flexibility of at least one of the first support member portion and the second support member portion.

In one example, the first and second support member portions are rotatably coupled to one another.

In one example, at least one of the first and second support member portions comprises a fluid conduit to direct the fluid to the respective nasal passage nozzle from the fluid source.

In one example, the animal intranasal administration device can further comprise at least one nostril nozzle configured to direct fluid onto the nostrils.

In one example, the animal intranasal administration device can further comprise a muzzle nozzle configured to direct fluid onto a muzzle.

In one example, the animal intranasal administration device can further comprise a positioning member configured to contact a tip of the septum to facilitate and maintain proper positioning of nasal passage nozzles.

In one example, the nasal passage nozzles are oriented to align nozzle openings with nasal passages when the device is engaged with the septum.

In one example, the nasal passage nozzles are configured to direct fluid into the nasal passages past nasal folds.

In one example, the nasal passage nozzles are configured to extend into the nostrils beyond the nasal folds.

In one example, the nasal folds comprise at least one of an alar fold, a basal fold, and a straight fold.

In one example, each of the nasal passage nozzles is configured to couple with a conduit to receive fluid from the fluid source.

In one example, the animal intranasal administration device can further comprise a fluid distribution manifold fluidly coupled to the nasal passage nozzles, the fluid distribution manifold having an inlet port to receive fluid from the fluid source and outlet ports to distribute fluid to the nasal passage nozzles.

In one example, the animal intranasal administration device can further comprise a septum interface portion associated with each of the nasal passage nozzles to interface with the septum and position the nasal passage nozzles to facilitate directing fluid into the nasal passages.

In one example, the animal intranasal administration device can further comprise a user interface to facilitate movement of the nasal passage nozzles by a user in a direction opposite a biasing direction.

In one example, the fluid is selected from the group consisting of: a liquid, a gas, a gel, or a combination thereof.

In one example, an animal intranasal administration device can comprise a support member having a first support member portion and a second support member portion, a first nasal passage nozzle coupled to the first support member portion, and a second nasal passage nozzle coupled to the second support member portion, wherein the first support member portion and the second support member portion are movable relative to one another to secure the first and second nasal passage nozzles at least partially within nostrils of an animal about a septum and such that fluid is directed into nasal passages of the animal.

In one example, the animal intranasal administration device can further comprise a first nostril nozzle and a second nostril nozzle coupled to the support member and configured to direct fluid onto the nostrils of the animal.

In one example, the support member comprises a first lateral extension portion and a second lateral extension portion to position the first nostril nozzle and the second nostril nozzle, respectively.

In one example, the first lateral extension portion and the second lateral extension portion are coupled to and extend from the first support member portion and the second support member portion, respectively.

In one example, the animal intranasal administration device can further comprise a muzzle nozzle coupled to the support member and configured to direct fluid onto a muzzle of the animal.

In one example, the muzzle nozzle is supported by at least one of the first and second support member portions.

In one example, the first and second support member portions are biased toward a secured position about the septum.

In one example, the animal intranasal administration device can further comprise a spring to bias the first and second support member portions toward the secured position.

In one example, at least one of the first and second support member portions is resiliently flexible to bias the at least one of the first and second support member portions toward the secured position.

In one example, the first and second support member portions are rotatably coupled to one another.

In one example, the support member is configured to provide clearance about a tip of the septum.

In one example, the first and second support member portions comprise arcuate configurations to provide clearance about the tip of the septum.

In one example, the animal intranasal administration device can further comprise a positioning member coupled to the support member and configured to contact a tip of the septum to facilitate and maintain proper positioning of first and second nasal passage nozzles within the nostrils of the animal.

In one example, the positioning member comprises an elongated portion having a longitudinal axis that is substantially parallel to an axis of rotation for movement of the first and second support member portions relative to one another.

In one example, the first and second nasal passage nozzles are oriented to align nozzle openings with the nasal passages of the animal when the device is engaged with the animal.

In one example, the first and second nasal passage nozzles are configured to direct fluid into the nasal passages past nasal folds.

In one example, the first and second nasal passage nozzles are configured to extend into the nostrils beyond the nasal folds.

In one example, the nasal folds comprise at least one of an alar fold, a basal fold, and a straight fold.

In one example, each of the first and second nasal passage nozzles is configured to couple with a conduit to receive fluid from a fluid source.

In one example, the fluid conduits are external to the support member.

In one example, at least one of the first and second support member portions comprises at least a portion of the conduit.

In one example, the animal intranasal administration device can further comprise a fluid distribution manifold fluidly coupled to the first and second nasal passage nozzles, the fluid distribution manifold having an inlet port to receive fluid from a fluid source and at least two outlet ports to distribute fluid to the first and second nasal passage nozzles.

In one example, the fluid distribution manifold is coupled to the support member.

In one example, the animal intranasal administration device can further comprise a septum interface portion associated with each of the first and second nasal passage nozzles to interface with the septum and position the nasal passage nozzles to facilitate directing fluid into the nasal passages.

In one example, the animal intranasal administration device can further comprise a user interface coupled to the support member to facilitate movement of the first and second support member portions relative to one another by a user.

In one example, the user interface comprises a first user interface portion coupled to the first support member portion, and a second user interface portion coupled to the second support member portion, and wherein the first and second user interface portions are movable relative to one another to facilitate movement of the first and second support member portions relative to one another.

In one example, the fluid is selected from the group consisting of: a liquid, a gas, a gel, or a combination thereof.

In one example, an animal intranasal administration system can comprise an animal intranasal administration device including a support member having a first support member portion and a second support member portion, a first nasal passage nozzle coupled to the first support member portion, and a second nasal passage nozzle coupled to the second support member portion, wherein the first support member portion and the second support member portion are movable relative to one another to secure the first and second nasal passage nozzles at least partially within nostrils of an animal about a septum and such that fluid is directed into nasal passages of the animal, and a fluid source fluidly coupled to each of the first and second nasal passage nozzles.

In one example, the animal intranasal administration system can further comprise a pump operable to deliver fluid from the fluid source to the first and second nasal passage nozzles.

In one example, the pump is configured to pump at least one of a liquid and a gas.

In one example, the pump comprises a motorized pump, a hand pump, or a combination thereof.

In one example, the fluid source is portable by a user while in use.

In one example, the animal intranasal administration system can further comprise a carrying strap coupleable to the fluid source to facilitate portability by the user.

In one example, the animal intranasal administration system can further comprise a fluid conduit to deliver fluid from the fluid source to the first and second nasal passage nozzles.

In one example, the fluid source comprises activated nitric oxide releasing solution.

In one example, the fluid source comprises inactivated nitric oxide releasing solution.

In one example, the fluid source comprises a container with the inactivated nitric oxide releasing solution disposed therein, and wherein the inactivated nitric oxide releasing solution is activatable within the container.

In one example, fluid is configured to dispense from the fluid source to the first and second nasal passage nozzles following activation of the nitric oxide releasing solution due to a pressure in the container resulting from the activation of the nitric oxide releasing solution.

In one example, the animal intranasal administration system can further comprise a cage for containing an activation agent prior to mixing the activation agent with the inactivated nitric oxide releasing solution, wherein the cage is configured to facilitate mixing of the activation agent and the inactivated nitric oxide releasing solution.

In one example, the cage is supported within the container above a bottom of the container.

In one example, the fluid source further comprises an activation agent maintained separate from the inactivated nitric oxide releasing solution and configured to activate the inactivated nitric oxide releasing solution upon mixing.

In one example, the fluid source is fluidly coupled to the first and second nasal passage nozzles via a first conduit associated with the inactivated nitric oxide releasing solution and a second conduit associated with the activation agent.

In one example, the first and second conduits combine prior to the first and second nasal passage nozzles such that mixing of the inactivated nitric oxide releasing solution and the activation agent occurs between the fluid source and the first and second nasal passage nozzles.

In one example, the first and second conduits combine at the first and second nasal passage nozzles such that mixing of the inactivated nitric oxide releasing solution and the activation agent occurs at the first and second nasal passage nozzles.

In one example, the first and second conduits remain separate from the fluid source to the first and second nasal passage nozzles such that mixing of the inactivated nitric oxide releasing solution and the activation agent occurs at the animal.

In one example, the fluid source comprises nitric oxide gas.

In one example, the animal comprises a domesticated animal.

In one example, the domesticated animal comprises a bovine, a swine, an equine, an ovine, or a goat.

In one example, a method of administering a fluid to an animal's nostril can comprise providing an animal intranasal administration device including a support member having a first support member portion and a second support member portion, a first nasal passage nozzle coupled to the first support member portion, and a second nasal passage nozzle coupled to the second support member portion, wherein the first support member portion and the second support member portion are movable relative to one another to secure the first and second nasal passage nozzles at least partially within nostrils of an animal about a septum and such that fluid is directed into nasal passages of the animal, engaging the device with the animal's nostril, and dispensing the fluid from the device and into the animal's nostrils.

In one example, the fluid is selected from the group consisting of: a liquid, a gas, a gel, or a combination thereof.

In one example, the fluid source comprises activated nitric oxide releasing solution.

In one example, an amount of nitric oxide releasing solution dispensed to the animal is between about 0.1 mL and about 5000 mL.

In one example, the amount of nitric oxide releasing solution dispensed to the animal is between about 10 mL and 1000 mL.

In one example, an amount of nitric oxide releasing solution dispensed to the animal is about 2 mL.

In one example, an amount of nitric oxide releasing solution dispensed to the animal is about 10 mL.

In one example, an amount of nitric oxide releasing solution dispensed to the animal is about 32 mL.

In one example, an amount of nitric oxide releasing solution dispensed to the animal is 160 mL.

In one example, the fluid source comprises inactivated nitric oxide releasing solution.

In one example, the method can further comprise activating the inactivated nitric oxide releasing solution.

In one example, the fluid is dispensed utilizing a gas pressure resulting from the activation of the nitric oxide releasing solution.

In one example, activating the inactivated nitric oxide releasing solution occurs prior to dispensing the fluid from the device and into the animal's nostril.

In one example, activating the inactivated nitric oxide releasing solution occurs when dispensing the fluid from the device and into the animal's nostril.

In one example, activating the inactivated nitric oxide releasing solution occurs after dispensing the fluid from the device and into the animal's nostril.

In one example, the fluid source comprises nitric oxide gas.

In one example, the animal comprises a domesticated animal.

In one example, the domesticated animal comprises a bovine, a swine, an equine, an ovine, or a goat.

In one example, the first and second nasal passage nozzles are configured to direct fluid into the nasal passages past nasal folds.

In one example, the first and second nasal passage nozzles are configured to extend into the nostrils beyond the nasal folds.

In one example, the nasal folds comprise at least one of an alar fold, a basal fold, and a straight fold.

It is noted that no specific order is required in the methods disclosed herein, though generally in some embodiments, the method steps can be carried out sequentially.

Of course, it is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. An animal intranasal administration device for a veterinary subject, comprising:
a nasal passage nozzle for a nostril configured to receive fluid from a fluid source;
a support structure opposing the nasal passage nozzle; and
a biasing mechanism to bias the nasal passage nozzle and the support structure toward a septum such that the device is secured in place about the septum during administration of the fluid into a nasal passage.

2. The animal intranasal administration device of claim 1, wherein the support structure comprises a second nasal passage nozzle.

3. The animal intranasal administration device of claim 1, wherein the biasing mechanism comprises a spring to bias the nasal passage nozzle and the support structure toward the septum.

4. The animal intranasal administration device of claim 1, further comprising a support member having a first support member portion in support of the nasal passage nozzle and a second support member portion in support of the support structure, wherein the first support member portion and the second support member portion are movable relative to one another by the biasing mechanism.

5. The animal intranasal administration device of claim 4, wherein the biasing mechanism comprises a spring acting on the first support member portion and the second support member portion.

6. The animal intranasal administration device of claim 4, wherein the biasing mechanism comprises resilient flexibility of at least one of the first support member portion and the second support member portion.

7. The animal intranasal administration device of claim 4, wherein the first and second support member portions are rotatably coupled to one another.

8. The animal intranasal administration device of claim 4, wherein the first support member portion comprises a fluid conduit to direct the fluid to the nasal passage nozzle from the fluid source.

9. The animal intranasal administration device of claim 1, further comprising at least one nostril nozzle configured to direct fluid onto the nostrils.

10. The animal intranasal administration device of claim 1, further comprising a muzzle nozzle configured to direct fluid onto a muzzle.

11. The animal intranasal administration device of claim 1, further comprising a positioning member configured to contact a tip of the septum to facilitate and maintain proper positioning of the nasal passage nozzle.

12. The animal intranasal administration device of claim 1, wherein the nasal passage nozzle is oriented to align a nozzle opening with the nasal passage when the device is engaged with the septum.

13. The animal intranasal administration device of claim 1, wherein the nasal passage nozzle is configured to direct fluid into the nasal passage past a nasal fold.

14. The animal intranasal administration device of claim 13, wherein the nasal passage nozzle is configured to extend into the nostril beyond the nasal fold.

15. The animal intranasal administration device of claim 13, wherein the nasal fold comprises at least one of an alar fold, a basal fold, and a straight fold.

16. The animal intranasal administration device of claim 1, wherein the nasal passage nozzle is configured to couple with a conduit to receive fluid from the fluid source.

17. The animal intranasal administration device of claim 2, further comprising a fluid distribution manifold fluidly coupled to the nasal passage nozzles, the fluid distribution manifold having an inlet port to receive fluid from the fluid source and outlet ports to distribute fluid to the nasal passage nozzles.

18. The animal intranasal administration device of claim 2, further comprising a septum interface portion associated with each of the nasal passage nozzles to interface with the septum and position the nasal passage nozzles to facilitate directing fluid into the nasal passages.

19. The animal intranasal administration device of claim 1, further comprising a user interface to facilitate movement of the nasal passage nozzle and the support structure by a user in a direction opposite a biasing direction.

20. The animal intranasal administration device of claim 1, wherein the fluid is selected from the group consisting of: a liquid, a gas, a gel, or a combination thereof.

21-101. (canceled)
